(19) United States
(12) Patent Application Publication
(10) Pub. No.: US 2009/0304757 A1
(43) Pub. Date: Dec. 10, 2009

(54) Cosmetic Compositions Comprising A Powdered Thermoplastic

(75) Inventors: Pascal Herve, Lyon (FR); Minou Nabavi, Paris (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: RHODIA OPERATIONS, AUBERVILLIERS (FR)

(21) Appl. No.: 12/373,242

(22) PCT Filed: Jul. 6, 2007
(86) PCT No.: PCT/EP07/56894

§ 371 Date: Jun. 24, 2009

(30) Foreign Application Priority Data
Jul. 11, 2006 (FR) 06 06300

(51) Int. Cl. A61K 8/02; A61K 8/84; A61K 8/90; A61Q 19/00; A61Q 5/00

(52) U.S. Cl. 424/401; 514/772.3; 514/772.1

(57) ABSTRACT

The present invention relates to cosmetic compositions used in particular in the care and makeup field. The invention relates to cosmetic compositions for making up and/or caring for keratin materials such as the skin, lips, nails, head hair, eyelashes, eyebrows or body hair of human beings. It relates more particularly to cosmetic compositions comprising a powdered thermoplastic polymer, and also to the use of this powder for improving the properties of cosmetic compositions.

The present invention relates to cosmetic compositions used in particular in the care and makeup field.

The invention relates to cosmetic compositions for making up and/or caring for human keratinous substances, such as the skin, lips, nails, hair, eyelashes, eyebrows or nonscalp hairs. It relates more particularly to cosmetic compositions comprising a powder made of thermoplastic polymer and to the use of this powder in improving the properties of cosmetic compositions.

Cosmetic compositions have to meet numerous requirements in the care and/or makeup field. They must, of course, exhibit the cosmetic properties desired according to their field of application (for example, protective properties with regard to the skin for a sun cream, and the like). However, another goal is good esthetic properties, such as an appearance and/or feel, and the like. Other properties are also desired, such as good adhesion, homogeneous and ready application, good hold of the composition on keratinous substances, and the like.

It is known to introduce thermoplastic polymer powders into cosmetic compositions, which makes it possible to improve some properties described above, such as the appearance.

It is a continual goal to improve the properties (in particular the properties described above) of cosmetic compositions.

To this end, the invention provides a cosmetic composition comprising a powder made of thermoplastic material P, the particles of the powder comprising an additive A formed by a polymeric material comprising at least a portion of its structure compatible with said thermoplastic material P and at least a portion of its structure incompatible with and insoluble in said thermoplastic material P.

According to a specific embodiment of the invention, the additive A is present at the surface of the powder particles.

According to another specific embodiment of the invention, the powder particles are present in a solid or liquid or pasty phase with which the incompatible portion of the additive A is compatible.

According to another characteristic of the invention, the additive A is advantageously a polymer of the block, sequence, comb, hyperbranched or star type. Thus, the structure compatible with the thermoplastic material forms a block, a sequence, the backbone or the teeth of the comb, or the core or the branches of the star or hyperbranched polymer.

According to a preferred embodiment of the invention, the compatible structure of the additive A comprises functional groups chemically identical to those of the thermoplastic polymer P.

According to the preferred embodiment of the invention, the additive A is chosen from the group consisting of a polymer D defined below or a hyperbranched polymer E comprising at least one polyalkylene oxide block.

Said polymer D is a polymer having thermoplastic properties comprising a block of thermoplastic polymer and at least one block of polyalkylene oxide, such that:
the block of thermoplastic polymer comprises a star or H macromolecular chain comprising at least one polyfunctional core and at least one branch or one segment of thermoplastic polymer connected to the core, the core comprising at least three identical reactive functional groups,
the block or blocks of polyalkylene oxide are connected to at least a portion of the free ends of the star or H macromolecular chain chosen from the thermoplastic polymer branch or segment ends and the ends of the polyfunctional core.

Such thermoplastic polymers and their process of preparation are described in particular in the document WO 03/002668, incorporated by reference.

The star macromolecular chain of the polymer D is advantageously a star polyamide obtained by copolymerization starting from a mixture of monomers comprising:
a) a polyfunctional compound comprising at least three identical reactive functional groups chosen from the amine functional group and the carboxylic acid functional group,
b) monomers of following general formulae (IIa) and/or (IIb):

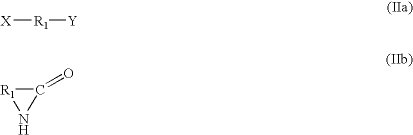

c) if appropriate, monomers of following general formula (III);

Z-R₂-Z (III)

in which:
Z represents a functional group identical to that of the reactive functional groups of the polyfunctional compound,
R₁ and R₂ represent identical or different, substituted or unsubstituted and aliphatic, cycloaliphatic or aromatic hydrocarbon radicals comprising from 2 to 20 carbon atoms which can comprise heteroatoms,
Y is a primary amine functional group when X represents a carboxylic acid functional group, or
Y is a carboxylic acid functional group when X represents a primary amine functional group.

The H macromolecular chain of the block of thermoplastic polymer of the polymer D is advantageously an H polyamide obtained by copolymerization starting from a mixture of monomers comprising:
a) a polyfunctional compound comprising at least three identical reactive functional groups chosen from the amine functional group and the carboxylic acid functional group,
b) lactams and/or amino acids,
c) a difunctional compound chosen from dicarboxylic acids or diamines,
d) a monofunctional compound, the functional group of which is either an amine functional group or a carboxylic acid functional group,
   the functional groups of c) and d) being amine when the functional groups of a) are acid, the functional groups of c) and d) being acid when the functional groups of a) are amine, the ratio as equivalents of the functional groups of a) to the sum of the functional groups of c) and d) being between 1.5 and 0.66, and the ratio as equivalents of the functional groups of c) to the functional groups of d) being between 0.17 and 1.5.

Advantageously, the polyfunctional compound of the star or H macromolecular chains is represented by the formula (IV):

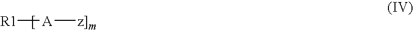

in which:
R1 is a linear or cyclic and aromatic or aliphatic hydrocarbon radical comprising at least two carbon atoms which can comprise heteroatoms,
A is a covalent bond or an aliphatic hydrocarbon radical comprising from 1 to 6 carbon atoms,
Z represents a primary amine radical or a carboxylic acid radical,
m is an integer between 3 and 8.

Preferably, the polyfunctional compound is chosen from 2,2,6,6-tetra(β-carboxyethyl)cyclohexanone, trimesic acid, 2,4,6-tri(aminocaproic acid)-1,3,5-triazine or 4-aminoethyl-1,8-octanediamine.

The block of polyalkylene oxide (POA) of the polymer D is preferably linear. It can be chosen from polyethylene oxide, polytrimethylene oxide or polytetramethylene oxide blocks. In the case where the block is based on polyethylene oxide, it can comprise propylene glycol units at the ends of the block. The block of polyalkylene oxide of the polymer D is preferably a block of polyethylene oxide.

Advantageously, all the free ends of the macromolecular chain of the block of thermoplastic polymer of the polymer D are connected to a block of polyalkylene oxide.

The term “hyperbranched polymer E according to the invention” is understood to mean a branched polymeric structure obtained by polymerization in the presence of compounds having a functionality of greater than 2 and having a structure which is not fully controlled. Random copolymers are often involved. The hyperbranched polymers can, for example, be obtained by reaction between, in particular, polyfunctional monomers, for example trifunctional and bifunctional monomers, each of the monomers carrying at least two different polymerization reactive functional groups.

Advantageously, the hyperbranched polymer E of the invention is chosen from hyperbranched polyesters, polyesteramides and polyamides.

The hyperbranched polymer E of the invention is preferably a hyperbranched copolyamide of the type of those obtained by reaction between:
at least one monomer of following formula (I):

A-R—B_{f} (I)
in which A is a polymerization reactive functional group of a first type, B is a polymerization reactive functional group of a second type which is capable of reacting with A, R is a hydrocarbon entity and f is the total number of B reactive functional groups per monomer: f≧2, preferably 2≦f≦10;
at least one monomer of following formula (II); (II) A′-R′—B′ or the corresponding lactams, in which A′, B′ and R′ have the same definition as that given above for A, B and R respectively in the formula (I);
at least one “core” monomer of following formula (III) or at least one “chain-limiting” monomer of following formula (IV);

R¹(B″)ₙ (III)

in which:
R¹ is a substituted or unsubstituted hydrocarbon radical of the silicone, linear or branched alkyl, aromatic, alkylaryl, arylalkyl or cycloaliphatic type which can comprise unsaturations and/or heteroatoms;
B″ is a reactive functional group of the same nature as B or B′;
n≧1, preferably 1≦n≦100;

R²-A″ (IV)

in which:
R² is a substituted or unsubstituted hydrocarbon radical of the silicone, linear or branched alkyl, aromatic, arylalkyl, alkylaryl or cycloaliphatic type which can comprise one or more unsaturations and/or one or more heteroatoms;
and A″ is a reactive functional group of the same nature as A or A′;
the molar ratio I/II being defined as follows;

0.05<I/II

and preferably 0.125≦I/II≦2;
at least one of the entities R or R′ of at least one of the monomers (I) or (II) being aliphatic, cycloaliphatic or arylaliphatic;
R₁ and/or R₂ being polyoxyalkylene radicals.

Such copolyamides are described in the document WO 00/68298 A1, incorporated by reference, in particular on page 11, lines 3 to 6.

The polymerization reactive functional groups A, B, A′ and B′ are advantageously chosen from the group consisting of carboxyl and amine functional groups.

The monomer of formula (I) of the hyperbranched copolyamide is advantageously a compound in which A represents the amine functional group, B represents the carboxyl functional group, R represents an aromatic radical and f=2.

R₁ and/or R₂ are advantageously amine-comprising polyoxyalkylene radicals of Jeffamine® type.

The proportion of additive A in the powder is advantageously between 0.01 and 40% by weight, with respect to the weight of the powder. This proportion of additive can be determined by an analytical method, such as by NMR or IR, preferably by proton NMR.

The powder of the invention can be produced from any thermoplastic material.

Mention may be made, as example of thermoplastic polymer, of polyamides, polyesters, polyurethanes, polyolefins, such as polyethylene or polypropylene, polystyrene, and the like.

According to a specific embodiment of the process of the invention, the preferred thermoplastic polymers are polyamides.

Any polyamide known to a person skilled in the art can be used in the context of the invention. The polyamide is generally a polyamide of the type of those obtained by polycondensation starting from dicarboxylic acids and diamines, or of the type of those obtained by polycondensation of lactams and/or amino acids. The polyamide of the invention can be a blend of polyamides of different types and/or of the same type, and/or copolymers obtained from different monomers corresponding to the same type and/or to different types of polyamide.

Mention may be made, as example of a polyamide which may be suitable for the invention, of polyamide 6, polyamide 6,6, polyamide 11, polyamide 12, polyamides 4,6, 6,10, 6,12, 12,12, and 6,36, semiaromatic polyamides, for example polyphthalamides obtained from terephthalic and/or isophthalic acid, such as the polyamide sold under the trade name Amodel, their copolymers and their alloys.

According to a preferred embodiment of the invention, the polyamide is chosen from polyamide 6, polyamide 6,6, their blends and their copolymers.

According to a specific embodiment of the invention, the thermoplastic polymer is a polymer comprising star macromolecular chains. The polymers comprising such star macromolecular chains are described, for example, in the documents FR 2 743 077, FR 2 779 730, U.S. Pat. No. 5,959,069, EP 0 632 703, EP 0 682 057 and EP 0 832 149. These compounds are known to exhibit an improved flowability in comparison with linear polyamides of the same molecular weight.

According to another specific embodiment of the invention, the thermoplastic polymer is a polycondensate composed of:
30 to 100 mol % (limits included) of macromolecular chains corresponding to the following formula (I):

R₃—(X—R₂—Y)ₙ—X-A-R₁-A-X—(Y—R₂—X)ₘ—R₃ (I)
0 to 70 mol % (limits included) of macromolecular chains corresponding to the following formula (II):

Rᵣ—[Y—R₂—X]ₚ—R₃ (II)

in which:
—X—Y— is a radical resulting from the polycondensation of two reactive functional groups F₁ and F₂, such that:
F₁ is the precursor of the radical —X— and F₂ is the precursor of the radical —Y—, or vice versa,
the functional groups F₁ cannot react with one another by condensation,
the functional groups F₂ cannot react with one another by condensation,
A is a covalent bond or an aliphatic hydrocarbon radical which can comprise heteroatoms and which comprises from 1 to 20 carbon atoms,
R₂ is a branched or unbranched aliphatic or aromatic hydrocarbon radical comprising from 2 to 20 carbon atoms,
R₃ and R₄ represent hydrogen, a hydroxyl radical or a hydrocarbon radical,
R₁ is a linear or cyclic and aromatic or aliphatic hydrocarbon radical comprising at least 2 carbon atoms which can comprise heteroatoms,
n, m and p each represent a number between 50 and 500, preferably between 100 and 400.

Such a polycondensate is described in application WO 05/019510, incorporated by reference. Advantageously, the polycondensate is a polyamide composed of;
30 to 100 mol % (limits included) of macromolecular chains corresponding to the following formula (I):

R₃—(X—R₂—Y)ₙ—X-A-R₁-A-X—(Y—R₂—X)ₘ—R₃ (I)
0 to 70 mol % (limits included) of macromolecular chains corresponding to the following formula (II):

Rᵣ—[Y—R₂—X]ₚ—R₃ (II)

in which:
Y is the

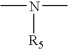

radical when X represents the

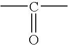

radical,
Y is the

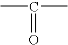

radical when X represents the

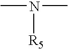

radical,
A is a covalent bond or an aliphatic hydrocarbon radical which can comprise heteroatoms and which comprises from 1 to 20 carbon atoms,
R₂ is a branched or unbranched aliphatic or aromatic hydrocarbon radical comprising from 2 to 20 carbon atoms,
R₃ and R₄ represent hydrogen, a hydroxyl radical or a hydrocarbon radical comprising a

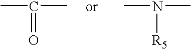

group,
R₅ represents hydrogen or a hydrocarbon radical comprising from 1 to 6 carbon atoms,
R₁ is a linear or cyclic and aromatic or aliphatic hydrocarbon radical comprising at least 2 carbon atoms which can comprise heteroatoms,
n, m and p each represent a number between 50 and 500, preferably between 100 and 400.

The thermoplastic polymers used in the invention can comprise various additives, such as matifying agents, heat stabilizers, light stabilizers, pigments, dyes and fillers, in particular abrasive fillers. Mention may in particular be made, by way of example, of titanium oxide, zinc oxide, cerium oxide, silica or zinc sulfide, which are used as matifying agent and/or abrasive.

The process of the invention can employ one or more additives A.

According to a specific embodiment of the invention, the particles of the powder of the invention are obtained according to the process comprising the following stages:
a. forming a melt blend of the thermoplastic material P with at least the additive A described above in order to obtain a dispersion of discrete particles of the thermoplastic material P,
b. cooling said blend to a temperature below the softening temperature of the thermoplastic material P,
c. treating said cooled blend in order to bring about separation of the discrete particles of thermoplastic material P.

The blend can be formed by melting the thermoplastic material P and adding the additive A in the solid or molten form and applying a blending energy in order to obtain the formation of the discrete particles of thermoplastic material.

The blend can also be formed by blending particles of said thermoplastic material P and particles of said additive A in the solid state and melting the blend of particles with application to the melt of a blending energy in order to obtain formation of discrete particles of thermoplastic material.

The concentration by weight of additive A in the blend is advantageously between 1% and 50%.

The melt blend can be shaped before the cooling stage.

The shaping process can be a process of extrusion through a die.

The melt blend can be produced in an extruder feeding the extrusion die.

The cooling can be pneumatic cooling. The cooling can also be obtained by dipping in a liquid.

The treatment for separation of the particles made of thermoplastic material P can be obtained by application of a shear force to the cooled blend.

The treatment for separation of the particles made of thermoplastic material P can also be obtained by dipping the cooled melt blend in a liquid which is not a solvent for the thermoplastic material P. This liquid is advantageously a solvent for the additive A.

Advantageously, the particles of the powder of the invention are spherical particles.

The term “spherical particle” is understood to mean a example of oily medium, of volatile or nonvolatile silicone oils, of liquid paraffin, of vegetable oil, of a wax, of glycerol, of ethylene glycol, and the like.

The cosmetic composition can comprise active agents, according to the application desired.

The Composition can-also comprise other additives, such as antioxidants, fragrances, preservatives, neutralizing agents, surfactants, film-forming polymers, thickeners, ultraviolet radiation blockers, vitamins, coloring materials, emulsion stabilizers, moisturizing agents, self-tanning compounds, antiwrinkle active agents, and the like.

The cosmetic composition of the invention can be prepared according to any method known to a person skilled in the art.

The composition according to the invention is advantageously a composition for making up and/or caring for keratinous substances, in particular a composition for caring for the face (cream or fluid), a composition for caring for the body (moisturizing, slimming), a water-resistant (waterproof) or non-water-resistant sun cream composition or a composition for making up the skin, such as a foundation, an eyeshadow, a blusher, a concealer or a product for making up the body.

According to a specific embodiment of the invention, the composition is chosen from the following compositions:
a composition for making up the eyes or the skin,
a composition for making up or protecting the lips,
a composition for caring for the skin,
a composition for protecting from the sun,
a composition for coating the nails,
a composition for cleaning the skin and/or hair intended to be rinsed out, in particular a shampoo, a shower gel, a product for cleaning the face or a conditioner intended to be rinsed out,
a composition for treating the hair which is not intended to be rinsed out, in particular a conditioner,
a composition for dyeing the hair,
a composition for shaping the hair (styling mousse, gels, gums, fixing sprays).

The powder made of thermoplastic material as described above makes it possible to improve the feel and/or the appearance of cosmetic compositions.

It can also improve the properties of adsorption/desorption of active materials, the resistance to abrasion and/or the adhesion of cosmetic compositions.

The composition of the invention can be easily and flexibly formulated.

According to a first specific embodiment of the invention, the cosmetic composition of the invention is an emulsion mainly comprising an aqueous phase and a fatty phase and additionally comprising the powder described above.

The use in these cosmetic compositions according to the invention of surfactants, of thickeners and more generally of surface-active agents makes it possible to obtain a stable dispersion of one phase in the other. It is also possible to have additives, such as preservatives and fragrances, but also cosmetic active principles, such as moisturizing agents (polyols), UV inhibitors, antiwrinkle active agents, self-tanning agents, film-forming agents, antioxidants and many others, in these compositions.

The compositions of continuous fatty phase type exhibit numerous advantages at the cosmetic and formulation level. The fatty phase according to the invention comprises solid or liquid fatty substances of vegetable, mineral, animal or synthetic origin. Mention may be made, for example, of esters, fatty alcohols, fatty acids or hydrocarbons essentially comprising carbon and hydrogen atoms and optionally nitrogen or oxygen atoms. This is because these formulations are advantageously compatible with the skin and its lipid constituents. The formation of a film at the surface of the skin contributes to limiting evaporation of water present in the layers of the skin and to keeping the latter moisturized, thus providing good protection of the skin with regard to drying out. Mention may also be made of silicone oils and fluorinated oils.

Furthermore, as the active principles (moisturizing agents, chemical or physical UV screening agents) are generally lipophilic, they can be more easily dispersed or dissolved in the continuous fatty phase which will carry them at the constituent layers of the skin, providing good distribution of these active principles. The continuous and hydrophobic fatty phase also constitutes a protective medium for these active principles. This is because body fluids (tears, perspiration) or water have a tendency to remove these active principles from the surface of the skin by washing or trickling. The incompatibility of the water with the continuous fatty phase prevents or greatly restricts this removal. This is particularly appreciable for skin-protecting suncreams of waterproof type which have to retain their protective effectiveness with regard to UV radiation even after bathing.

The composition of the emulsion is advantageously as follows;
from 10 to 75%, preferably from 30 to 65%, of an aqueous phase;
from 0.1 to 30%, preferably from 1 to 20%, of the powder described above; and
from 89.9 to 24.9% of a fatty phase, the total forming 100%.

The fatty phase advantageously comprises less than 25% (by weight with respect to the total composition) of volatile oil. The volatile oil is advantageously a silicone oil. The aqueous phase advantageously comprises from 10 to 60% of polyols.

Fatty Phase

A fatty phase can comprise a liquid fatty phase and optionally a solid fatty phase (such as waxes). The liquid fatty phase can comprise one or more oils which are liquid at ambient temperature (25° C.); these oils are volatile or nonvolatile. The liquid fatty phase is formed of hydrocarbon oils, indeed even optionally of silicone oils.

The fatty phase of the emulsion is a continuous fatty phase which, with the water, provides an emulsion in the water-in-oil form. This fatty phase comprises one or more oils, that is say water-immiscible fatty substances. These volatile or nonvolatile oils are of mineral, animal, vegetable or synthetic origin and can be hydrocarbon, silicone or fluorinated oils. The term “hydrocarbon oil” is understood to mean an oil formed essentially, indeed even composed, of carbon and hydrogen atoms and optionally of oxygen or nitrogen atoms. It can comprise alcohol, ester, ether, carboxylic acid, amine and/or amide groups. It can comprise one or more oils which are liquid at ambient temperature (25° C.), preferably at least one nonvolatile liquid oil. The term “nonvolatile liquid oil” is understood to mean an oil which is capable of remaining on the skin at ambient temperature (25° C.) and atmospheric pressure for at least one hour and which has in particular a nonzero vapor pressure at ambient temperature (25° C.) and atmospheric pressure of less than or equal to 0.01 mmHg (1.33 Pa).

The liquid fatty phase advantageously comprises one or more nonvolatile oils which provide an emollient effect on the skin. Mention may be made of fatty esters, such as cetearyl isononanoate, isotridecyl isononanoate, isostearyl isostearate, isopropyl isostearate, isopropyl myristate, isopropyl palmitate, butyl stearate, hexyl laurate, isononyl isononanoate, 2-ethylhexyl palmitate, 2-hexyldecyl laurate, 2-octyl-decyl palmitate, 2-octyldodecyl myristate or lactate, di(2-ethylhexyl) succinate, diisostearyl malate, glyceryl or triglyceryl triisostearate, or tocopheryl acetate, higher fatty acids, such as myristic acid, palmitic acid, stearic acid, behenic acid, oleic acid, linoleic acid, linolenic acid or isostearic acid, caprylic/capric acid triglyceride, higher fatty alcohols, such as oleyl alcohol, avocado oil, camellia oil, macadamia nut oil, turtle oil, mink oil, soybean oil, grape seed oil, sesame oil, corn oil, rapeseed oil, sunflower oil, cottonseed oil, jojoba oil, peanut oil, olive oil, hexyl laurate and their mixtures. They can be mineral oils: hydrocarbon oils, such as liquid paraffin, squalane, liquid petrolatum and their mixtures.

The emulsion optionally comprises nonvolatile silicone oils, such as, for example, dimethylsiloxanes.

The liquid fatty phase can also optionally comprise volatile oils. The term “volatile oil” is understood as meaning an oil capable of evaporating from the skin in less than one hour at ambient temperature and atmospheric pressure. This oil has in particular a vapor pressure at ambient temperature (25° C.) and atmospheric pressure (760 mmHg) of greater than 0.01 and less than or equal to 300 mmHg (1.33 Pa to 40 000 Pa) and preferably ranging from 0.05 to 300 mmHg (6.65 Pa to 40 000 Pa).

The volatile oils are chosen, for example, from silicone oils which contribute to reducing the greasy effect of formulations with a continuous fatty phase. Mention may be made of linear or cyclic silicone oils having a viscosity at ambient temperature of less than 8 mm²/s and having in particular from 2 to 7 silicon atoms, these silicones optionally comprising alkyl or alkoxy groups having from 1 to 10 carbon atoms. Mention may be made, as volatile silicone oil which can be used in the invention, of in particular octamethylcyclo-tetrasiloxane, decamethylcyclopentasilqxane, dodeca-methylcyclohexasiloxane, heptamethylhexyltrisiloxane, heptamethyloctyltrisiloxane, hexamethyldisiloxane, octamethyltrisiloxane, decamethyltetrasiloxane, dodeca-methylpentasiloxane and their mixtures. They are more particularly from the family of the polyalkyl- or polyarylsiloxanes: cyclomethicone (DC 345 from Dow Corning), caprylyl methicone or cyclopentasiloxane (DC245 from Dow Corning). Mention may also be made of volatile hydrocarbon oils having from 8 to 16 carbon atoms and their mixtures and in particular branched C₈ to C₁₆ alkanes, such as C₈ to C₁₆ isoalkanes (also referred to as isoparaffins), isododecane, isodecane, isohexadecane, branched C₈ to C₁₆ esters, such as isohexyl neopentanoate, and their mixtures.

Advantageously, the emulsion -comprises at most 25% of volatile oil and in particular of volatile silicone oil, preferably at most 15% (% by weight, with respect to the total composition).

Aqueous Phase

The aqueous phase comprises water. The latter can be a floral water, such as cornflower water, and/or a mineral water, such as water from Vittel, water from Lucas or water from La Roche Posay, and/or a thermal water. The aqueous phase can also comprise water-miscible constituents, such as, for example, primary alcohols, such as ethanol and isopropanol, polyols, such as glycols added for their humectant properties: glycerol, propylene glycol, butylene glycol, dipropylene glycol or diethylene glycol, glycol ethers, such as mono-, di- or tripropylene glycol or mono-, di- or triethylene glycol (C₁-C₄)alkyl ethers, and their mixtures. The aqueous phase can additionally comprise stabilizing agents, such as sodium chloride, magnesium dichloride and magnesium sulfate. The aqueous phase can also comprise any water-soluble or water-dispersible compound compatible with an aqueous phase, such as gelling agents, film-forming polymers, thickeners, surfactants and their mixtures.

Other Compounds

The emulsion according to the invention can also comprise surfactants (generally lipophilic) of anionic, nonionic or amphoteric type which facilitate the dispersion of the aqueous phase in the fatty phase, so as to obtain a stable water/oil emulsion, or additives, such as preservatives (generally hydrophilic), fragrances (generally lipophilic), fillers other than the powder according to the invention, coloring materials (pigments, soluble dyes), thickeners (waxes, gelling agents), emulsion stabilizers (generally hydrophilic) or chelating agents (generally hydrophilic). The surfactants can be of ester type, such as sorbitan derivatives (e.g., sorbitan sesqui-isostearate) or methyl glucose isostearate. They can be of polymer type, such as PEG-45/dodecyl glycol copolymer. They can also be silicone surfactants suitable for the emulsification of silicone oils: they are, for example, cimethicone copolyols, such as PEG/PPG-18/18 dimethicone, sold by Dow Corning under the name DC5225C. The thickeners can, for example, be soluble in the fatty phase in order to adjust its consistency or to contribute to the stability of the composition: mention may be made, for example, of candelilla wax or silicone gums or elastomers (DC1411 and DC9040 from Dow Corning). The preservatives are mixtures of paraben derivatives and/or of phenoxyethanol. Mention may be made, for example, of EthyleneDiamineTetraAcetic (EDTA) as chelating agent. They can also comprise cosmetic active principles which improve the human keratinous substances mentioned above. The cosmetic active principles comprise moisturizing agents (generally hydrophilic), such as polyols, UV radiation blockers, such as organic screening agents (generally lipophilic) or inorganic particles, such as TiO₂ or ZnO, which may or may not be surface treated, antiwrinkle active agents (generally hydrophilic), self-tanning agents (generally hydrophilic), film-forming agents (lipophilic or hydrophilic, depending on their nature) or antioxidants (lipophilic or hydrophilic, depending on their nature). Mention may be made, as inorganic screening agents, of dispersions of ZnO and of TiO₂ in mixtures of silicone oils.

The emulsion according to the invention advantageously comprises:
from 10 to 75%, preferably from 30 to 65%, of aqueous phase;
from 0.1 to 30%, preferably from 1 to 20%, of powder according to the invention; and
from 89.9 to 24.9% of fatty phase, the total forming 100% (% by weight).

For its part, the aqueous phase preferably comprises from 10 to 60% of polyols, with respect to the total aqueous phase. Furthermore, it can comprise from 0.5 to 10%, preferably from 3 to 5%, of surfactants, from 0.01 to 2% of additives and from 0.005 to 10% of cosmetic active principles, with respect to the total composition.

The powder of the invention contributes greatly to the soft feel and to the obtaining, after application, of the mat and powdered appearance of the composition, is involved in the absorption of a portion of the fatty phase and contributes to limiting the negative effects of said fatty phase (greasy, oily or sticky feeling).

According to a second specific embodiment of the invention, the cosmetic composition is intended to be applied and rinsed out. Shampoos or shower gels or conditioners may be involved, for example.

The composition intended to be rinsed out comprises a cosmetically acceptable carrier, preferably water, optionally a surfactant and the powder of the invention.

The composition of the invention intended to be rinsed out exhibits good properties in terms of viscosity, transparency or deposition of material (conditioning effect, deposition of a polymer carrying cationic charges or deposition of other materials, such as mineral, vegetable or synthetic oils, for example silicone oils or “polyorganosiloxanes”) and/or more generally exhibits good cosmetic properties, such as softness, suppleness, disentangling, sheen or ability to be styled, on dry or wet hair. The composition in addition exhibits satisfactory foaming properties, in particular in hard water. This composition is also, a formulation which is easy to prepare, easy to use and satisfactorily stable.

Cosmetically Acceptable Carrier

Any cosmetically acceptable carrier which makes it possible to formulate the powder and to obtain the cosmetic composition form intended to be rinsed out desired for the targeted use can be used. Various carriers cosmetically acceptable for various types of formulation are known to a person skilled in the art.

Mention may be made, as examples of cosmetically acceptable carriers, of aqueous carriers (comprising water), alcoholic carriers (comprising an alcohol, for example ethanol, isopropanol, ethylene glycol, propylene glycol or polyethylene glycols) or aqueous/alcoholic carriers (comprising a mixture of water and of an alcohol, for example ethanol, isopropanol, ethylene glycol or polyethylene glycols). Certain volatile or nonvolatile oils can also be used. Mention is made, for example, of fluid silicones, such as cyclopentasiloxane, for example Mirasil CM5, sold by Rhodia.

A person skilled in the art knows to choose the carriers suited to the types of formulation desired and to the uses targeted. For example, aqueous carriers are generally used for shampoos or shower gels. A propylene glycol carrier can be used for compositions in the form of creams. A cyclomethicone carrier can be used for makeup compositions, for example for foundations.

Composition

Surfactants

The composition intended to be rinsed out is an aqueous composition optionally comprising surfactants. A mixture of different surfactants may be involved. The surfactants included in the composition preferably comprise at least one anionic surfactant. The surfactants can also comprise amphoteric (true amphoteric or zwitterionic) surfactants, neutral surfactants (nonionic surfactants) and/or cationic surfactants. The compositions comprising at least one anionic surfactant and at least one amphoteric surfactant are particularly advantageous, in particular for reasons of softness. The total content of surfactants in the composition is between 0 and 30% by weight.

For compositions intended for the treatment of the hair, such as shampoos, the content of surfactant is advantageously between 10 and 20% by weight. Such compositions can comprise salts, for example sodium chloride or ammonium chloride, advantageously in a content of less than 3% by weight.

For compositions intended for the treatment of the skin, such as shower gels, the content of surfactant is advantageously between 5 and 15% by weight. Such compositions also preferably comprise at least 2% by weight of salts, for example sodium chloride or ammonium chloride.

For conditioners, the content of surfactants can be less than 5% by weight.

The proportion by weight of anionic surfactants, with respect to the combined surfactants, is preferably greater than 50%, preferably greater than 70%.

Parameters (pH)

The pH of the composition is advantageously greater than or equal to 3.5. It is, for example, between 3.5 and 9, preferably between 5 and 7. The pH obviously depends on the compounds present in the composition. It is obviously possible to use, in the composition, pH-regulating agents, which are acids or bases, for example citric acid or sodium hydroxide, potassium hydroxide or ammonium hydroxide.

Natures of the Surfactants

The anionic surfactants can be chosen from the following surfactants:
alkyl ester sulfonates, for example of formula R—CH(SO₃M)—CH₂COOR′, or alkyl ester sulfates, for example of formula R—CH(OSO₃M)—CH₂COOR′, where R represents a C₈-C₂₀, preferably C₁₀-C₁₆, alkyl radical, R′represents a C₁-C₆, preferably C₁-C₃, alkyl radical and M represents an alkaline earth metal cation, for example sodium, or the ammonium cation. Mention may very particularly be made of methyl ester sulfonates in which the radical R is a C₁₄-C₁₆ radical;
alkylbenzenesulfonates, more particularly C₉-C₂₀ alkylbenzenesulfonates, primary or secondary alkylsulfonates, in particular C₈-C₂₂ alkylsulfonates, or alkylglycerolsulfonates:
alkyl sulfates, for example of formula ROSO₃M, where R represents a C₁₀-C₂₄, preferably C₁₂-C₂₀, alkyl or hydroxyalkyl radical and M represents a cation with the same definition as above;
alkyl ether sulfates, for example of formula RO(OA)ₙSO₃M, where R represents a C₁₀-C₂₄, preferably C₁₂-C₂₀, alkyl or hydroxyalkyl radical; OA represents an ethoxylated and/or propoxylated group; M represents a cation with the same definition as above and n varies generally from 1 to 4, such as, for example, lauryl ether sulfate with n=2;
alkylamide sulfates, for example of formula RCONHR′OSO₃M, where R represents a C₂-C₂₂, preferably C₆-C₂₀, alkyl radical, R′ represents a C₂-C₃ alkyl radical and M represents a cation with the same definition as above, and their polyalkoxylated (ethoxylated and/or propoxylated) derivatives (alkylamido ether sulfates);
salts of saturated or unsaturated fatty acids, for example such as C₈-C₂₄, preferably C₁₄-C₂₀, fatty acids, and of an alkaline earth metal cation, N-acyl-N-alkyltaurates, alkylisethionates, alkyl-succinamates and alkylsulfosuccinates, monoesters or diesters of sulfosuccinates, N-acylsarcosinates or polyethoxycarboxylates;
phosphate mono- and diesters, for example of the following formula: (RO)ₓ—P(═O)(OM)ₓ, where R represents an optionally polyalkoxylated alkyl, alkylaryl, arylalkyl or aryl radical, x and x′ are equal to 1 or 2, provided that the sum of x and x′ is equal to 3, and M represents an alkaline earth metal cation.

The nonionic surfactants can be chosen from the following surfactants:
alkoxylated fatty alcohols:
alkoxylated triglycerides;
alkoxylated fatty acids;
alkoxylated sorbitan esters;
alkoxylated fatty amines;
alkoxylated di(1-phenylethyl)phenols;
alkoxylated tri(1-phenylethyl)phenols;
alkoxylated alkylphenols;
the products resulting from the condensation of ethylene oxide with a hydrophobic compound resulting from the condensation of propylene oxide with propylene glycol, such as the Pluronic products sold by BASF;
the products resulting from the condensation of ethylene oxide with the compound resulting from the condensation of propylene oxide with ethylene-diamine, such as the Tetronic products sold by BASF,
alkylpolyglycosides, such as those described in U.S. Pat. No. 4,565,647;
fatty acid amides, for example C₈-C₂₀ fatty acid amides.

The amphoteric surfactants (true amphoteric, comprising an ionic group and a potentially ionic group of opposite charge, or zwitterionic, simultaneously comprising two opposite charges) can be chosen from the following surfactants:
betaines generally, in particular carboxybetaines, for example lauryl betaine (Mirataine BB from Rhodia) or octyl betaine, or amidoalkyl betaines, such as cocamidopropyl betaine (CAFB) (Mirataine BDJ from Rhodia Chimie);
sulfobetaines or sultaines, such as cocamidopropyl hydroxysultaine (Mirataine CBS from Rhodia);
alkylamphoacetates and alkylamphodiacetates, such as, for example, comprising a coco or lauryl chain (Miranol C2M, C32 and L32 in particular from Rhodia),
alkylamphopropionates or alkylamphodipropionates (Miranol C2M SF);
alkyl amphohydroxypropyl sultaines (Miranol CS).

The cationic surfactants can be chosen from salts of optionally polyethoxylated primary, secondary or tertiary fatty amines, quaternary ammonium salts, such as tetraalkylammonium, alkylamidoalkylammonium, trialkylbenzylammonium, trialkylhydroxyalkylammonium or alkylpyridinium chlorides or bromides, imidazoline derivatives or amine oxides having a cationic nature.

Mention may be made, as examples of useful compositions, of:
“Sodium” compositions for shampoos typically comprising 12 to 16% by weight of sodium alkyl ether sulfate (for example, sodium lauryl ether sulfate “SLES”) or of a mixture of sodium alkyl ether sulfate and of sodium alkyl sulfate (for example, sodium lauryl sulfate “SLS”), 1 to 3% of an amphoteric surfactant (for example, cocamidopropyl betaine “CAPB”) and 0.5 to 2% of a salt (for example, sodium chloride).
“Ammonium” compositions for shampoos typically comprising 12 to 16% by weight of ammonium alkyl ether sulfate (for example, ammonium lauryl ether sulfate “ALES”) or of a mixture of ammonium alkyl ether sulfate and of ammonium alkyl sulfate (for example, ammonium lauryl sulfate “ALS”), 1 to 3% of an amphoteric surfactant (for example, cocamidopropyl betaine “CAPB”) and 0 to 2% of a salt (for example, ammonium chloride).
“Sodium” compositions for shower gels typically comprising 6 to 10% by weight of sodium alkyl ether sulfate (for example, sodium lauryl ether sulfate “SLES”) or of a mixture of sodium alkyl ether sulfate and of sodium alkyl sulfate (for example, sodium lauryl sulfate “SLS”), 1 to 3% of an amphoteric surfactant (for example, cocamidopropyl betaine “CAPB”) and 2 to 4% of a salt (for example, sodium chloride).
“Ammonium” compositions for shower gels typically comprising 6 to 10% by weight of ammonium alkyl ether sulfate (for example, ammonium lauryl ether sulfate “ALES”) or of a mixture of ammonium alkyl ether sulfate and of ammonium alkyl sulfate (for example, ammonium lauryl sulfate “ALS”), 1 to 3% of an amphoteric surfactant (for example, cocamidopropyl betaine “CAPB”) and 0 to 4% of a salt (for example, ammonium chloride).

Other Compounds

The composition intended to be rinsed out can comprise any other compound used in cosmetic compositions intended to be rinsed out (shampoo, shower gel, conditioner, and the like).

Mention is made, for example, of sequestering agents, softening agents, foam modifiers, colorants, pearlescent agents (pearlizers), moisturizing agents, antidandruff or antiseborrheic agents, suspending agents, emulsifying agents, ceramides, pseudoceramides, electrolytes, fatty acids, fatty acid esters, hydroxy acids, thickening agents, fragrances, preservatives, organic or inorganic sunscreens, proteins, vitamins, polymers, silicones (polyorganosiloxanes), or stabilizing and/or conditioning agents and/or conditioning aid, other than the polyorganosiloxanes, in particular polymers. Some of these compounds are described in detail below.

Stabilizing and/or Conditioning Agent and/or Conditioning Aid

The cosmetic composition intended to be rinsed out according to the invention can advantageously comprise at least one stabilizing and/or conditioning agent and/or conditioning aid. The term “suspending agents” is also sometimes used. The term “conditioning aid” is understood to mean that the presence of the agent improves the conditioning related to other compounds, for example oils or silicones. The agents are understood as agents other than the polyorganosiloxane of formula (I). Such agents are known to a person skilled in the art. The composition according to the invention can comprise several of these agents (mixtures or combinations), in order to combine their effects and/or to create synergies. Furthermore, some agents can play several roles. This is the case, for example, of polysaccharides and their cationic derivatives, for example cationic guar derivatives.

The proportion by weight of such agents can be typically from 0.1% to 10% by weight, preferably from 0.3% to 8% by weight, for polysaccharides or other agents.

Mention may be made, as examples of stabilizing agents which are particularly useful for compositions comprising polyorganosiloxanes, of:
crosslinked polyacrylates, for example polymers of Carbopol or Carbomer type sold by BF Goodrich or Noveon, Acritamer sold by Rita or Tego Carbomer sold by Goldschmidt. These compounds can typically be present in an amount of 0.1 to 3% by weight, preferably of 0.3 to 2% by weight, with respect to the composition;
C₁₀-C₃₀ alkyl PEG 20 acrylate/aminoacrylate/itaconate copolymers sold by National Starch under the name Structure Plus. These compounds can typically be present in an amount of 0.1 to 3% by weight, preferably of 0.3 to 2% by weight, with respect to the composition;
insoluble solids forming a network in the composition. They can be ethylene glycol mono- and/or diesters of fatty acids, the fatty acids preferably being C₁₆-C₁₈ fatty acids. The solid can in particular be ethylene glycol distearate (EGDS), for example sold by Rhodia as a concentrate with other ingredients under the name Mirasheen. This compound can typically be present in an amount of 3 to 10% by weight, preferably of 5 to 8% by weight, with respect to the composition.

Mention may also be made of viscosifying, gelling or texturing agents, such as the anionic acrylic copolymers of Aculyne type sold by ISP or Rohm & Haas), or polysaccharides and their noncationic derivatives, such as cellulose derivatives, for example hydroxy-propylcellulose or carboxymethylcellulose, nonionic guar derivatives, such as hydroxypropyl guar (for example, the Jaguar HP product sold by Rhodia), locust bean gum, tara gum or cassia gum, xanthan gum (for example, the Rhodicare product sold by Rhodia), succinoglycans (for example, Rheozan sold by Rhodia), alginates, carrageenans, chitin derivatives or any other polysaccharide having a texturing role. These polysaccharides and their derivatives can be incorporated alone or in synergistic combination with other polysaccharides. These compounds can typically be present in an amount of 0.1 to 3% by weight, preferably of 0.3 to 1% by weight, with respect to the composition.

Mention may be made, as examples of stabilizing agents and/or of conditioning agents and/or of conditioning aids, of:
cationic polymers derived from polysaccharides, for example cationic cellulose derivatives, catibnic starch derivatives, cationic guar derivatives or cationic locust bean derivatives;
synthetic cationic polymers;
mixtures or combinations of these agents.

The synthetic or nonsynthetic cationic polymers which can act as conditioning agent are in particular polymers of polyquaternium type, such as, for example, polyquaternium-1, polyquaternium-2, polyquaternium-4, polyquaternium-5, polyquaternium-6 (also known as merquat 1000, available from Nalco), polyquaternium-7 (also known as Merquat 5500, available from Nalco), polyquaternium-8, polyquaternium-9, polyquaternium-10 (also known as Polymer JR 400, sold by Amercol), polyquaternium-11, polyquaternium-12, poly-quaternium-13, polyquaternium-14, polyquaternium-15, polyquaternium-16, polyquaternium-17, poly-quaternium-18, polyquaternium-19, polyquaternium-20, polyquaternium-22 (also known as Merquat 280, Merquat 281 or Merquat 298, available from Nalco), polyquaternium-24, polyquaternium-27, polyquaternium-28, polyquaternium-29 (also known as Kytamer KCO, available from Amerchol), polyquaternium-30, polyquaternium-31, polyquaternium-32 poly-quaternium-33, polyquaternium-34, pQlyquaternium-35, polyquaternium-36, polyquaternium-37, polyquaternium-39 (also known as Merquat 3300 or Merquat 3331, available from Nalco), polyquaternium-44, polyquaternium-27 (also known as Merquat 2001, available from Nalco) and polyquaternium-55.

As mentioned above, the composition intended to be rinsed out can comprise other polymers, synthetic or natural or resulting from biological preparation processes, if appropriate functionalized, for example by cationic or neutral groups. These polymers can have a stabilizing or structuring effect on the compositions and/or a conditioning effect (deposition at the surface of the skin or hair).

Mention is made, as examples, of cationic polysaccharide derivatives, such as guar or cellulose derivatives. Cationic polymers functionalized by hydrophobic groups, such as C₁-C₁₄, preferably C₂-C₈, alkyl chains, optionally exhibiting a hydroxyl group, can be used. These hydrophobic groups are attached to the main polymer chain via ether bonds.

Furthermore, in the case of hydrophobically or non-hydrophobically modified cationic guars, the cationic group is a quaternary ammonium group carrying three identical or different radicals chosen from hydrogen or an alkyl radical comprising from 1 to 22 carbon atoms, more particularly from 1 to 14 carbon atoms and advantageously from 1 to 3 carbon atoms. The counterion is a halogen, preferably chlorine.

In the case of hydrophobically or nonhydrophobically modified cationic celluloses, the cationic group is a quaternary ammonium group carrying three identical or different radicals chosen from hydrogen or an alkyl radical comprising from 1 to 10 carbon atoms, more particularly from 1 to 6 carbon atoms and advantageously from 1 to 3 carbon atoms. The counterion is a halogen, preferably chlorine.

Mention may be made, among cationic guar derivatives, of guar hydroxypropyltrimonium chloride (Jaguar C13S, Jaguar C14S, Jaguar C17, Jaguar Excel or Jaguar C 2000, sold by Rhodia Chimie) or hydroxypropyl guar hydroxypropyltrimonium chloride (Jaguar C162, sold by Rhodia).

Use can be made, among cationic cellulose derivatives, of cellulose [2-hydroxy-3-{trimethylammonio}propyl]-poly(oxy-1,2-ethanediyl) ether chloride or polyquaternium-10, such as Polymer JR400 (INPI name: PQ10), sold by Amerchol.

Nonionic polysaccharide derivatives, for example hydroxypropyl guar, can also be used.

Synthetic polymers, more particularly homopolymers, such as polymethacrylamidopropyl trimonium chloride (Polycare 133, sold by Rhodia Chimie), may likewise be suitable.

The cationic polymers more particularly exhibit a weight-average molar mass of at least 2000 g/mol and more preferably of between 2×10⁴ and 3×10⁶ g/mol, according to their degree of polymerization possible. The weight-average molar masses of the polymers are usually measured by size exclusion. They can optionally be measured directly by light scattering or from the intrinsic viscosity using calibration according to “Viscosity-Molecular weight relationship, intrinsic chain flexibility and dynamic solution properties of guar galactomannan” by G. Robinson, S. B. Ross Murphy and E. R. Morris, Carbohydrate Research, 107, pp 17-32, 1982.

In the case of cationic polysaccharide derivatives, the degree of hydroxyalkylation (molar substitution or MS) is preferably between 0 and 1.2. Still in the case of these polymers, the degree of cationicity (degree of substitution or DS) is more particularly between 0.01 and 0.6. This is the case, for example, of Jaguar C162 and Jaguar C2000, sold by Rhodia Chimie.

Polyorganosiloxanes (Silicones)

The composition intended to be rinsed out can comprise a silicone (silicone oil). The term “silicone” or “polyorganosiloxane” is understood to mean any organosiloxane compound comprising alkyl (for example methyl) groups and/or functionalized by groups other than alkyl groups.

The polyorganosiloxane is advantageously (in shampoos and conditioners in particular) a nonvolatile and water-insoluble polyorganosiloxane. It advantageously exhibits a viscosity of between 1000 and 2 000 000 mpa·s, preferably between 5000 and 500 000 mpa·s. The polyorganosiloxane can in particular be a polydimethylorganosiloxane (“PDMS”, INCI name: dimethicone), or a polyorganosiloxane exhibiting amine groups (for example, amodimethicone according to the INCI name), quaternary ammonium groups (for example, silicone quaternium-1 to -10 according to the INCI name), hydroxyl groups (terminal or nonterminal), polyoxyalkylene groups, for example polyethylene oxide and/or polypropylene oxide groups (as terminal groups, as blocks within a PDMS chain or as grafts), or several of these groups.

The amount of polyorganosiloxane present in the composition can typically be from 0.1% to 5% by weight, for example from 0.5% to 1.5% or 2% by weight.

The polyorganosiloxanes (silicones) are preferably present in the composition intended to be rinsed out in the emulsion form (liquid silicone droplets dispersed in the aqueous phase). The emulsion can in particular be an emulsion for which the mean size of the droplets is greater than or equal to 2 μm and/or for which the mean size of the droplets is greater than or between 0.15 μm and 2 μm or for which the mean size of the droplets is less than or equal to 0.15 μm.

The droplets of the emulsion can be more or less large in size. Reference may thus be made to microemulsions, to miniemulsions or to macroemulsions. In the present patent application, the term “emulsion” covers in particular all these types of emulsion. Without wishing to be committed to any one theory, it is specified that microemulsions are generally thermodynamically stable systems generally comprising large amounts of emulsifying agents. The other emulsions are generally systems in the non-thermodynamically stable state which retain for a certain time, in the metastable state, the mechanical energy provided during the emulsification. These systems generally comprise lesser amounts of emulsifying agents.

The emulsions can be obtained by mixing the carrier, preferably aqueous carrier, the polyorganosiloxane and generally an emulsifying agent, and then emulsifying. It is possible to speak of in situ emulsification.

The compositions intended to be rinsed out in the emulsion form can also be obtained by mixing the carrier, preferably aqueous carrier, with a preprepared emulsion of droplets comprising the polyorganosiloxane in an external phase which is preferably miscible with the cosmetically acceptable carrier, preferably of the same nature as said carrier, preferably an aqueous carrier. This embodiment may be preferred as it is simple to implement. In addition, this embodiment is particularly suitable for the implementation of cosmetic compositions in which the polyorganosiloxane is in the microemulsion form. It is possible to speak of preemulsification.

According to a specific embodiment, the emulsion is a microemulsion, the size of the droplets of which is less than 0.15 μm. In this embodiment, the composition preferably comprises a proportion of emulsifying agent of greater than 10% by weight, preferably at least 15% by weight, with respect to the weight of polyorganosiloxane.

The size of the microemulsion droplets can be measured on an emulsion prepared prior to its introduction into the cosmetic composition by dynamic light scattering (QELS), for example as described below. The equipment used is, for example, composed of a Spectra-Physics 2020 laser, of a Brookhaven 2030 correlator and of the associated computing. As the sample is concentrated, it is diluted in deionized water and filtered through a 0.22 μm filter in order, at the end, to be at 2% by weight. The diameter obtained is an apparent diameter. The measurements are carried out at angles of 90° and 135°. For the size measurements, in addition to the conventional analysis by cumulants, the autocorrelation function is run in three ways (the exponential sampling or EXPSAM described by Pr. Pike, the “Non Negatively Constrained Least Squares” or NNLS method and the CONTXN method described by Pr. Provencher) which each give a size distribution weighted by the scattered intensity and not by the weight or the number. The refractive index and the viscosity of the water are taken into account.

According to an advantageous form, the microemulsion is transparent. The microemulsion can, for example, exhibit a transmittance of at least 90%, preferably of at least 95%, at a wavelength of 600 nm, measured, for example, using a Lambda 40 UV-vis spectrometer at a concentration of 0.5% by weight in water. In this context, the cosmetic composition can advantageously be transparent. It can, for example, exhibit a transmittance of at least 90%, preferably of at least 95%, at a wavelength of 600 nm, measured, for example, using a Lambda 40 UV-Vis spectrometer.

According to another specific embodiment, the emulsion is an emulsion for which the mean size of the droplets is greater than or equal to 0.15 μm, for example greater than 0.5 μm, or than 1 μm, or than 2 μm, or than 10 μm, or than 20 μm, and preferably less than 100 μm. The size of the droplets can be measured, by optical microscopy and/or laser particle sizing (Horiba LA-910 laser scattering analyzer), on an emulsion prepared prior to its introduction into the cosmetic composition or directly on the cosmetic composition diluted in water. In this embodiment, the composition preferably comprises a proportion of emulsifying agent of less than 10% by weight, with respect to the weight of polyorganosiloxane.

Emulsifying agents of use in the preparation of polyorganosiloxane emulsions are in particular nonionic surfactants, preferably polyalkoxylated surfactants, for example chosen from alkoxylated fatty alcohols, alkoxylated triglycerides, alkoxylated fatty acids, alkoxylated sorbitan esters, alkoxylated fatty amines, alkoxylated di(1-phenylethyl)phenols, alkoxylated tri(1-phenylethyl)phenols and alkoxylated alkylphenols, where the number of alkoxy units, more particularly oxyethylene and/or oxypropylene units, is such that the HLB value is greater than or equal to 10.

Mention may be made, among the silicone derivatives which are soluble in the water of the composition, inter alia, of dimethicone copolyols (Mirasil DMCO, sold by Rhodia Chimie).

As relates to the silicones which are provided in the form of dispersions which are insoluble in the water of the composition, use may suitably be made of water-insoluble and nonvolatile organopolysiloxanes, among which may be mentioned polyalkylailoxane, polyarylsiloxane or polyalkylarylsiloxane oils, gums or resins or their water-insoluble functionalized derivatives, or their mixtures, which are nonvolatile.

Said organopolyosiloxanes are regarded as water-insoluble and nonvolatile if their solubility in water is less than 50 g/liter and their intrinsic viscosity is at least 3000 mpa·s at 25° C.

Mention may be made, as examples of water-insoluble and nonvolatile organopolysiloxanes or silicones, of silicone gums, such as, for example, the diphenyl dimethicone gum sold by Rhodia Chimie, and preferably the polydimethylsiloxanes exhibiting a viscosity at least equal to 6×10⁵ mpa·s at 25° C. and more preferably still those with a viscosity of greater than 2×10⁶ mpa·s at 25° C., such as Mirasil DM 500 000®, sold by Rhodia Chimie.

According to the invention, the water-insoluble and nonvolatile organopolysiloxane or silicone occurs in a form dispersed within the cosmetic composition including it.

The water-insoluble and nonvolatile organopolysiloxane or silicone exists in the form of particles or droplets, the size of which can be chosen according to the nature of the cosmetic composition or the performance desired for said composition. Generally, this size can vary from 0.01 to 70 microns.

Preferably, this size is of the order of 0.1 to 50 microns, very particularly of the order of 1 to 30 microns.

In order to facilitate the use thereof, these organopolysiloxanes can be dispersed or dissolved beforehand in volatile or nonvolatile silicone derivatives of low viscosity and then emulsified in the cosmetic composition.

Mention may be made, among these silicones of low viscosity, of volatile cyclic silicones and polydimethylsiloxanes of low weight.

Use can also be made of functionalized silicone derivatives, such as aminated derivatives, directly in the form of emulsions or starting from a preformed microemulsion. They can be compounds known under the term of aminated silicones or hydroxylated silicones. Mention is made of Mirasil ADM-E (amodimethicone), sold by Rhodia, and dimethiconol.

Mention is in particular made, as polyorganosiloxanes which can be used, of:
polyorganosiloxanes comprising —Si(CH₃O— units and —SiY(CH₃)O— units where Y is a —(CH₂)₃—NH(CH₂)₂—NH₂ or —(CH₂)₃—NH₂ group,
polyorganosiloxanes comprising —Si(CH₃)₂O— units and HO—Si(CH₃)₂O— terminal units and/or —Si(CH₃)(OH)O— nonterminal units,
polyorganosiloxanes comprising —Si(CH₃)₂O— units and —SiY(CH3)O— units where Y is -L^{x}-Z^{x}-Palc where L^{x} is a divalent connecting group, preferably an alkylene group, Z^{x} is a covalent bond or a divalent joining group comprising a heteroatom, Palc is a group of formula [OE]ₐ-[OP]ₜ—X′, in which OE is a group of formula —CH₂—CH₂—O—, OP is a group of formula —CH₂—CHCH₃—O— or —CHCH₃—CH₂—O—, X′ is a hydrogen atom or a hydrocarbon group, a is a mean number greater than 1 and t is a mean number greater than or equal to 0,
polyorganosiloxanes, the chain of which comprises at least one block comprising units of formula —Si(CH₃)₂O— units and at least one —[OE]ₛ—[OP]ₜ— block,
polyorganosiloxanes comprising —Si(CH₃)₂O— units and/or —Si(CH₃)RO— and/or —SiR₂O— and/or R—Si(CH₃)₂O— and/or H₃C—SiR₂O— - and/or R—SiR₂O— units, where R, which can be identical or different, is an alkyl group other than a methyl group, an aryl group, an alkylaryl group or an aralkyl group.

Other Compounds

It is likewise possible to envisage using oils which may perform conditioning, protective or emollient roles. Such oils are generally chosen from alkyl monoglycerides, alkyl diglycerides, triglycerides, such as oils extracted from plants and vegetables (palm oil, coconut oil, cottonseed oil, soybean oil, sunflower oil, olive oil, grape seed oil, sesame oil, peanut oil, castor oil, and the like) or oils of animal origin (tallow, fish oils, and the like), derivatives of these oils, such as hydrogenated oils, lanolin derivatives, petrolatum, mineral oils or liquid paraffins, perhydrosqualane, squalene, diols, such as 1,2-dodecanediol, cetyl alcohol, stearyl alcohol, oleyl alcohol, fatty esters, such as isopropyl palmitate, 2-ethylhexyl cocoate or myristyl myristate, lactic acid esters, stearic acid, behenic acid or isostearic acid.

It is also possible to incorporate bactericidal or fungicidal agents in the cosmetic composition intended to be rinsed out, in the form of dispersions or solutions, in order to improve the disinfecting of the skin, such as, for example, triclosan, antidandruff agents, such as, in particular, zinc pyrithione or octopyrox, or insecticidal agents, such as natural or synthetic pyrethroids.

The cosmetic compositions intended to be rinsed out can also comprise agents for protecting the skin and/or hair against attacks from the sun and UV radiation. Thus, the compositions can comprise sunscreens, which are chemical compounds which strongly absorb UV radiation, such as the compounds authorized in European Directive No. 76/768/EEC, its appendices and the subsequent amendments to this directive.

In the case where the various constituent components of the cosmetic composition intended to be rinsed out exhibit an excessively low solubility in the composition or when they exist in the solid form at ambient temperature, said constituent components can advantageously be dissolved in an organic vehicle, such as mineral or natural oils, silicone derivatives or waxes, or alternatively can be encapsulated in matrices, such as polymers of latex type.

The cosmetic compositions intended to be rinsed out forming the subject matter of the invention can also comprise fixative resins.

These fixative resins, when they are present, are generally present at concentrations of between 0.01 and 10%, preferably between 0.5 and 5%.

The fixative resins participating in the cosmetic compositions intended to be rinsed out are chosen more particularly from the following resins:
methyl acrylate/acrylamide copolymers, poly(vinyl methyl ether/maleic anhydride) copolymers, vinyl acetate/crotonic acid copolymers, octylacrylamide/methyl acrylate/butylaminoethyl methacrylate copolymers, polyvinylpyrrolidones, polyvinyl-pyrrolidone/methyl methacrylate copolymers, polyvinylpyrrolidone/vinyl acetate copolymers, polyvinyl alcohols, polylvinyl alcohol/crotonic acid) copolymers, poly(vinyl alcohol/maleic anhydride) copolymers, hydroxypropyl celluloses, hydroxypropyl guars, sodium polystyrenesulfonates, polyvinyl-pyrrolidone/ethyl methacrylate/methacrylic acid terpolymers, poly(methyl vinyl ether/maleic acid) monomethyl ethers or polyvinyl acetates grafted to polyoxyathylene backbones (EP-A-219 048),
copolyesters derived from terephthalic and/or isophthalic and/or sulfoisophthalic acid, anhydride or a diester thereof and from a diol, such as:
   polyester copolymers based on ethylene terephthalate and/or propylene terephthalate and polyoxyethylene terephthalate units (U.S. Pat. No. 3,959,230, U.S. Pat. No. 3,893,929, U.S. Pat. No. 4,116,896, U.S. Pat. No. 4,702,857, U.S. Pat. No. 4,770,666);
   sulfonated polyester oligomers obtained by sulfonation of an oligomer derived from ethoxylated allyl alcohol, from dimethyl terephthalate and from 1,2-propanediol (U.S. Pat. No. 4,968,451);
   polyester copolymers derived from dimethyl terephthalate, from isophthalic acid, from dimethyl sulfoisophthalate and from ethylene glycol (EP-A-540 374);
   copolymers comprising polyester units derived from dimethyl terephthalate, from isophthalic acid, from dimethyl sulfoisophthalate and from ethylene glycol and polyorganosiloxane units (FR-A-2 728 915);
   sulfonated polyester oligomers obtained by condensation of isophthalic acid, of dimethyl sulfosuccinate and of diethylene glycol (FR-A-2 236 926);
   polyester copolymers based on propylene terephthalate and polyoxyethylene terephthalate units and terminated by ethyl or methyl units (U.S. Pat. No. 4,711,730) or polyester oligomers terminated by alkylpolyethoxy groups (U.S. Pat No. 4,702,857) or anionic sulfopolyethoxy (U.S. Pat. No. 4,721,580) or sulfoaroyl (U.S. Pat. No. 4,877,896) groups;
   polyester-polyurethanes obtained by reaction of a polyester, obtained from adipic acid and/or from terephthalic acid and/or from sulfoisophthalic acid and from a diol, with a prepolymer comprising isocyanate terminal groups obtained from a polyoxyethylene glycol and from a diisocyanate (FR-A-2 334 698);
ethoxylated monoamines or polyamines, polymers of ethoxylated amines (U.S. Pat. No. 4,597,898, EP-A-11 989).

Preferably, the fixative resins are chosen from polyvinylpyrrolidones (PVP), copolymers of vinylpyrrolidone and of methyl methacrylate, copolymers of vinylpyrrolidone and of vinyl acetate (VA), poly(ethylene glycol terephthalate/ethylene glycol) copolymers, polyfethylene glycol terephthalate/ethylone glycol/sodium sulfoisophthalate) copolymers, and their blends.

These fixative resins are preferably dispersed or dissolved in the chosen vehicle.

The cosmetic compositions intended to be rinsed out forming the subject matter of the invention can also comprise polymeric derivatives performing a protective role.

These polymeric derivatives can be present in amounts of the order of 0.01-10% by weight, preferably approximately 0.1-5% by weight, and very particularly of the order of 0.2-3% by weight.

These agents can in particular be chosen from:
nonionic cellulose derivatives, such as cellulose hydroxyethers, methylcellulose, ethylcellulose, hydroxypropyl methylcellulose or hydroxybutyl methylcellulose;
polyvinyl esters grafted to polyalkylene backbones, such as polyvinyl acetates grafted to polyoxyethylene backbones (EP-A-219 048);
polyvinyl alcohols.

The cosmetic compositions intended to be rinsed out forming the subject matter of the invention can also comprise plasticizers.

Said plasticizers, if they are present, can represent between 0.1 and 20% of the formulation, preferably from 1 to 15%.

Mention may be made, among particularly useful plasticizers, of adipates, phthalates, isophthalates, azelates, stearates, silicone copolyols, glycols, castor oil or their mixtures.

It is also advantageously possible to add metal-sequestering agents to these compositions, more particularly those which sequester calcium, such as citrate ions.

It is also possible to incorporate humectants in the cosmetic compositions intended to be rinsed out forming the subject matter of the invention, which humectants include, inter alia, glycerol, sorbitol, urea, collagen, gelatin, aloe vera, hyaluronic acid or water-soluble volatile solvents, such as ethanol or propylene glycol, the contents of which can reach up to 60% by weight of the composition.

In order to further reduce irritation of or attack on the scalp, it is also possible to add water-soluble or water-dispersible polymers, such as collagen or some non-allergizing derivatives of animal or plant proteins (wheat protein hydrolysates, for example), natural hydrocolloids (guar gum, locust bean gum, tara gum, and the like) or hydrocolloids resulting from fermentation processes, and derivatives of these polycarbohydrates, such as nonionic modified celluloses, such as, for example, hydroxyethylcellulose, or anionic modified celluloses, such as carboxymethylcellulose, or guar or locust bean derivatives, such as their nonionic derivatives (for example, hydroxpropyl guar) or the anionic derivatives (carboxymethyl guar and carboxymethyl hydroxypropyl guar).

Inorganic powders of particles, such as calcium carbonate, sodium bicarbonate, calcium dihydrogenphosphate, inorganic oxides in the powder form or in the colloidal form (particles with a size of less than or of the order of a micrometer, sometimes of a few tens of nanometers), such as titanium dioxide or silica, aluminum salts, generally used as antiperspirants, kaolin, talc, clays and their derivatives, and the like, can be added to these compounds in combination.

Preservatives, such as the methyl, ethyl, propyl and butyl esters of p-hydroxybenzoic acid, sodium benzoate, Germaben® or any chemical agent which prevents proliferation of bacteria or molds and which is conventionally used in cosmetic compositions, can also be introduced into the aqueous cosmetic compositions according to the invention, generally at a level of 0.01 to 3% by weight.

The amount of these products is usually adjusted in order to prevent any proliferation of bacteria, molds or yeasts in the cosmetic compositions.

Alternatively to these chemical agents, it is sometimes possible to use agents which modify the activity of the water and which greatly increase the osmotic pressure, such as carbohydrates or salts.

In order to protect the skin and/or hair from attacks from the sun and UV radiation, it is possible to add organic or inorganic sunscreens to the compositions, for example inorganic particles, such as zinc oxide, titanium dioxide or cerium oxides, in the powder form or in the form of colloidal particles, alone or as a mixture. These powders can optionally be surface treated in order to enhance the effectiveness of their UV protective action or in order to facilitate their incorporation in the cosmetic formulations or in order to inhibit surface photoreactivity. The organic sunscreens can in particular be introduced into the polyorganosiloxane, if it is present in the composition.

If necessary, and with the aim of enhancing the comfort during use of the composition by the consumer, it is possible to add, to these ingredients, one or more fragrances, coloring agents, among which may be mentioned the products described in Appendix IV (“List of colouring agents allowed for use in cosmetic products”) of the European Directive No. 76/768/EEC of 27 Jul. 1976, known as the Cosmetics Directive, and/or opacifying agents, such as pigments.

Although not obligatory, the composition intended to be rinsed out can also comprise viscosifying or gelling polymers, so as to adjust the texture of the composition, such as crosslinked polyacrylates (Carbopol, sold by Goodrich), already mentioned above, noncationic cellulose derivatives, such as hydroxypropylcellulose or carboxymethylcellulose, guars and their nonionic derivatives, xanthan gum and its derivatives, used alone or in combination, or the same compounds, generally in the form of water-soluble polymers modified by hydrophobic groups covalently bonded to the polymer backbone, as described in patent WO 92/16187, and/or water, in-order to bring the total of the constituents of the formulation to 100%.

The cosmetic compositions intended to be rinsed out forming the subject matter of the invention can also comprise polymeric dispersing agents in an amount of the order of 0.1-7% by weight, in order to control the calcium and magnesium hardness, agents such as;
water-soluble salts of polycarboxylic acids with a weight-average molecular weight of the order of 2000 to 100 000 g/mol, obtained by polymerization or copolymerization of ethylenically unsaturated carboxylic acids, such as acrylic acid, maleic acid, maleic anhydride, fumaric acid, itaconic acid, aconitic acid, mesaconic acid, citraconic acid or methylenemalonic acid, and very particularly polyacrylates with a weight-average molecular weight of the order of 2000 to 10 000 g/mol (U.S. Pat. No. 3,308,067) or copolymers of acrylic acid and of maleic anhydride with a weight-average molecular weight of the order of 5000 to 75 000 g/mol (EP-A-66 915);
polyethylene glycols with a weight-average molecular weight of the order of 1000 to 50 000 g/mol.

Other details or advantages of the invention will become more clearly apparent in the light of the examples given below solely by way of indication.

### EXAMPLES

### Example 1 to 5

### Preparation of Powders

### Examples 1 to 4

The materials used in the examples are as follows:

Polymer P: polyamide 6,6 with a relative viscosity of 2.6

Additive A; hydrophilic star polyamide/polyalkylene oxide copolymer produced in the following way:

1116.0 g of ε-caprolactam (9.86 mol), 57.6 g of 1,3,5-benzenetricarboxylic acid (0.27 mol), 1826.4 g of Jeffamine® M2070 (0.82 mol), 1.9 g of Ultranox® 236 and 3.5 g of 50% (w/w) aqueous hypophosphorous acid solution are introduced into a 7.5 liter autoclave equipped with a mechanical stirrer.

The reaction mixture is brought to 250° C. under nitrogen and at atmospheric pressure and maintained at this temperature for 1 h. The system is then gradually placed under vacuum over 30 min down to a pressure of 5 mbar and then maintained under vacuum for an additional hour. The system is subsequently run onto a plate.

Compound B: Polyethylene oxide with a molecular weight of 1500 g/mol

The following are introduced into a 24D twin-screw extruder of Prism type: granules of polymer P using feeding by volume and a blend of pellets of the additive A and of compound B using feeding by weight. The throughputs of the two metering devices are adjusted so as to be able to vary the concentration of additive A and of compound B in the blend with the thermoplastic polymer P. The blends are extruded at a fixed throughput between 1.9 and 2.2 kg/hour. The temperatures of the various zones of the extruder are between 275 and 295° C. The speed is set at 200 rpm. The pressure recorded is between 10 and 13 bar. The rods obtained are quenched at the die outlet with a stream of water, collected in a metal basket, drained and then dried.

The rods collected are subsequently dispersed in water by simple mechanical stirring. The dispersion thus obtained is sieved with a 200 μm sieve to remove the large solid impurities, such as nondispersible pieces of rod. The yields by weight for recovery of thermoplastic polymer P after sieving are greater than 90%. The particle size distribution of the particles present in the dispersion is measured using a MasterSizer 2000 device sold by Malvern Instruments. This distribution, expressed by volume, obtained after application of ultrasound, is unimodal and the value reported in the tables below corresponds to the value of the modal peak.

The powder is subsequently washed 5 times by centrifuging and removing the supernatant, and the concentrations of residual additive A are measured by proton NMR.

Various powders, described in table 1 below, were obtained and characterized according to the procedure described above.

The percentages below are expressed by weight, with respect to the weight of the composition.

TABLE 1

| | | Additive | | Residual | |
|---|---|---|---|---|---|
| Example | Polymer P | A | Compound B | additive A | Size |
| No. | (%) | (%) | (%) | (%) | (μm) |
| 1 | 85.0 | 7.5 | 7.5 | 5 | 2.5 μm |
| 2 | 79.5 | 7.5 | 13.0 | 1.6 | 5.0 μm |
| 3 | 73.0 | 7.5 | 19.5 | 1.3 | 10.0 μm |
| 4 | 80.0 | 20.0 | — | 5.6 | 0.7 μm |

### Example 5

The materials used are as follows:

Polymer P: A star polyamide obtained by copolymerization from caprolactam in the presence of approximately 0.5 mol % of 2,2,6,6-tetra(β-carboxy-ethyl)cyclohexanone, according to a process described in the document FR 2 743 077.

Additive A: A triblock copolymer: polyethylene oxide-block-polypropylene oxide-block-polyethylene oxide: CAS No.: 9003-11-6; product sold by Aldrich (catalog reference No. 412325, weight Mn˜14 600 g/mol; PEG 82.5% by weight).

The following are introduced into a 34D twin-screw extruder of Leistritz type: granules of polymer P using feeding by volume and pellets of the additive A using feeding by weight. The throughputs of the two metering devices are adjusted so as to be able to vary the concentration of additive A in the blend with the thermoplastic polymer P. The blends are extruded at a throughput 20 kg/hour. The temperatures of the various zones of the extruder are between 230 and 240° C. The speed is set at 260 rpm. The pressure recorded is between 10 and 13 bar. The rods obtained are quenched at the die outlet with a stream of water, collected in a metal basket, drained and then dried.

The rods collected are subsequently dispersed in water by simple mechanical stirring. The dispersion thus obtained is sieved with a 200 μm sieve to remove the large solid impurities, such as nondispersible pieces of rod. The yields by weight for recovery of thermoplastic polymer P after sieving are greater than 98%. The particle size distribution of the particles present in the dispersion is measured using a MasterSizer 2000 device sold by Malvern Instruments. This distribution, expressed by volume, obtained after application of ultrasound, is unimodal and the value reported in the tables below corresponds to the value of the modal peak.

The powder is subsequently washed 7 times with simple separation by settling and removal of the supernatant, and the concentrations of residual additive A are measured by proton NMR.

A powder, described in table 1 below, was obtained and characterized according to the procedure described above.

The percentages below are expressed by weight, with respect to the weight of the composition.

TABLE 2

| | | | | Residual | |
|---|---|---|---|---|---|
| Example | Polymer P | Additive A | Compound B | additive A | Size |
| No. | (%) | (%) | (%) | (%) | (μm) |
| 5 | 72.5 | 27.5 | — | 1.7 | 45 μm |

### Examples 6 to 12

The preparation is carried out of shampoo compositions comprising ingredients chosen from the following in table 3 below:

TABLE 3

| Ingredient | Type | Compound |
|---|---|---|
| SLES | Anionic | Sodium lauryl ether sulfate (2 EO), |
| | surfactant | sold by Huntsman under the |
| | | reference Empicol ESB/3M ® |
| SLS | Anionic | Sodium lauryl sulfate |
| | surfactant | |
| ALES | Anionic | Ammonium lauryl ether sulfate |
| | surfactant | (2 EO), sold by Rhodia under the |
| | | reference Rhodapex-EA-2 ® |
| ALS | Anionic | Ammonium lauryl sulfate, sold by |
| | surfactant | Rhodia under the reference Rhodapon |
| | | L-22 ® |
| CAPB | Amphoteric | Cocamidopropyl betaine, sold by |
| | surfactant | Rhodia under the reference |
| | | Mirataine BET-C-30 ® |
| Salt | | Sodium chloride or ammonium |
| | | chloride |
| Polyamide | | Examples 1 to 5 |
| powder | | |
| Silicone 1 | Conditioning | Dimethicone (PDMS) emulsion with a |
| | agent | viscosity of approximately |
| | | 500 000 cP, with a size of the |
| | | droplets of approximately 2 μm, |
| | | stabilized by succinoglycan, sold |
| | | by Rhodia under the reference |
| | | Mirasil DME-2 ® |
| Silicone 2 | Conditioning | Dimethicone (PDMS) emulsion with a |
| | agent | viscosity of approximately |
| | | 500 000 cP, with a size of the |
| | | droplets of approximately 2 μm, |
| | | stabilized by succinoglycan, sold |
| | | by Rhodia under the reference |
| | | Mirasil DME-30 ® |

Procedure
1. The water and the polyamide powder are mixed. The mixture is subjected to ultrasound for 5 minutes.
2. The CAPB is added.
3. The anionic surfactant and then optionally the silicone emulsion are added.
4. The pH is adjusted to 6-6.5 by addition of sodium hydroxide or citric acid.
5. The salt is added.

The following shampoo compositions are produced, the proportion by weight of each ingredient of which is given in table 4 below:

TABLE 4

| | Example | | | | | | |
|---|---|---|---|---|---|---|---|
| | 6 | 7 | 8 | 9 | 10 | 11 | 12 |
| SLES (%) | 14 | 14 | 14 | 14 | 14 | 14 | 14 |
| SLS (%) | / | / | / | / | / | / | / |
| ALES (%) | / | / | / | / | / | / | / |
| ALS (%) | / | / | / | / | / | / | / |
| CAPB (%) | 2 | 2 | 2 | 2 | 2 | 2 | 2 |
| Salt: | / | / | / | / | / | | / |
| NH₄Cl (%) | | | | | | | |
| Salt: | 1.6 | 2.3 | 1.6 | 1.6 | 2 | 2 | 2.16 |
| NaCL (%) | | | | | | | |
| Polyamide | 0.5 | 0.5 | 0.5 | 0.5 | 0.5 | 0.5 | 0.5 |
| powder | (ex. | (ex. | (ex. | (ex. | (ex. | (ex. 2 | (ex. |
| (%) | 4) | 4) | 5) | 3) | 3) | | 1) |
| Citric | 0.15 | 0.34 | 0.09 | 0.17 | 0.09 | 0.35 | 0.14 |
| acid | | | | | | | |

| | |
|---|---|
| Water | Remainder to 100% |

The Brookfield viscosity of the compositions is measured using a Brookfield DV-I viscometer, at 22° C., 10 revolutions per minute, spindle 4 for viscosities of less than 11 000 mPa·s, spindle 5 for viscosities from 11 000 to 30 000 mPa·s and spindle 6 above these values.

| | Example | | | | | | |
|---|---|---|---|---|---|---|---|
| | 6 | 7 | 8 | 9 | 10 | 11 | 12 |
| Viscosity | 1820 | 23 680 | 1380 | 1300 | 11 800 | 11 360 | 11 340 |

### Example 13

0.5% or 1% by weight of polyamide powder according to example 3 is added to the transparent varnish sold by Gemey and the powder is dispersed using ultrasound. The varnish, thus modified, exhibits improved properties, in particular in terms of resistance to scratching.

1-30. (canceled)

31. A composition comprising a thermoplastic powder, wherein particles of the powder comprise a polymeric additive comprising at least a portion of its structure compatible with said thermoplastic powder, and at least a portion of its structure incompatible with and insoluble in said thermoplastic powder.

32. The composition of claim 31, wherein the polymeric additive is present at the surface of the powder particles.

33. The composition of claim 31, wherein the powder particles are present in a solid, or liquid, or pasty phase, with which the incompatible portion of the polymeric additive is compatible.

34. The composition of claim 31, wherein additive A is a block, sequence, comb, hyperbranched, or star polymer.

35. The composition of claim 34, wherein the structure compatible with the thermoplastic powder constitutes a block of a block polymer, a sequence of a sequential polymer, the teeth of a comb polymer, or the core or the branches of a star or hyperbranched polymer.

36. The composition of claim 31, wherein the compatible structure of the polymeric additive comprises functional groups identical to those of the thermoplastic powder.

37. The composition of claim 31, wherein the polymeric additive is a block copolymer D comprising a block of thermoplastic polymer and at least one block of polyalkylene oxide, wherein
the block of thermoplastic polymer comprises a star or H macromolecular chain comprising at least one polyfunctional core and at least one branch or one segment of thermoplastic polymer connected to the core, the core comprising at least three identical reactive functional groups; and
the at least one block of polyalkylene oxide is connected to at least a portion of the free ends of the star or H macromolecular chain chosen from the thermoplastic polymer branch or segment ends and the ends of the polyfunctional core.

38. The composition of claim 37, wherein the star macromolecular chain of the block of thermoplastic polymer of the polymer D is a star polyamide copolymer comprising:
a) a polyfunctional compound comprising at least three identical reactive functional groups chosen from the amine functional group and the carboxylic acid functional group,
b) monomers of following general formulae (IIa) and/or (IIb): 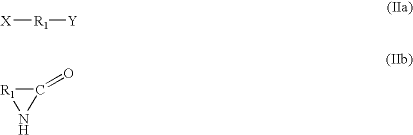
c) optionally, monomers of following general formula (III):
Z-R₂-Z (II)
in which:
Z represents a functional group identical to that of the reactive functional groups of the polyfunctional compound,
R₁ and R₂ represent identical or different, substituted or unsubstituted and aliphatic, cycloaliphatic or aromatic hydrocarbon radicals comprising from 2 to 20 carbon atoms which can comprise heteroatoms,
Y is a primary amine functional group when X represents a carboxylic acid functional group, or
Y is a carboxylic acid functional group when X represents a primary amine functional group.

39. The composition of claim 37, wherein the H macromolecular chain to the block of thermoplastic polymer of the polymer D is an H copolymer comprised of a mixture of monomers comprising:
a) a polyfunctional compound comprising at least three identical reactive functional groups chosen from the amine functional group and the carboxylic acid functional group,
b) lactams and/or amino acids,
c) a difunctional compound chosen from dicarboxylic acids or diamines,
d) a monofunctional compound, the functional group of which is either an amine functional group or a carboxylic acid functional group,
the functional groups of c) and d) being amine when the functional groups of a) are acid, the functional groups of c) and d) being acid when the functional groups of a) are amine, the ratio as equivalents of the functional groups of a) to the sum of the functional groups of c) and d) being between 1.5 and 0.66, and the ratio as equivalents of the functional groups of c) to the functional groups of d) being between 0.17 and 1.5.

40. The composition of claim 38 or 39, wherein the polyfunctional compound is represented by the formula (IV): 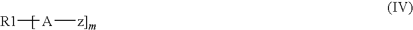
in which;
R₁ is a linear or cyclic and aromatic or aliphatic hydrocarbon radical comprising at least two carbon atoms which can comprise heteroatoms,
A is a covalent bond or an aliphatic hydrocarbon radical comprising from 1 to 6 carbon atoms,
Z represents a primary amine radical or a carboxylic acid radical,
m is an integer between 3 and 8.

41. The composition of claim 38 or 39, wherein the polyfunctional compound is 2,2,6,6-tetra(β-carboxyethyl) cyclohexanone, trimesic acid, 2,4,6-tri-(aminocaproic acid)-1,3,5-triazine, or 4-amino-ethyl-1,8-octanediamine.

42. The composition of claim 37, wherein the block of polyalkylene oxide of the polymer D is linear.

43. The composition of claim 42, wherein the block of polyalkylene oxide of the polymer D is a block of polyethylene oxide.

44. The composition of claim 37, wherein the free ends of the macromolecular chain of the block of thermoplastic polymer of the polymer D are connected to a block of polyalkylene oxide.

45. The composition of claim 35, wherein the hyperbranched polymer E is polyesters, polyesteramides or polyamides.

46. The composition of claim 35, wherein the hyperbranched polymer E is a hyperbranched copolyamide comprising:
at least one monomer of following formula (I):
A-R—B_{f} (I)
in which A is a polymerization reactive functional group of a first type, B is a polymerization reactive functional group of a second type which is capable of reacting with A, R is a hydrocarbon entity and f is the total number of B reactive functional groups per monomer: f≧2;
at least one monomer of following formula (II):
A′-R′—B′ or the corresponding lactams, (II)
in which A′, B′ and R′ have the same definition as that given above for A B and R respectively in the formula (I);
at least one “core” monomer of following formula (III) or at least one “chain-limiting” monomer of following formula (IV):
R¹(B″)ₙ (III)
in which
R¹ is a substituted or unsubstituted silicone, linear or branched alkyl, aromatic, alkylaryl, arylalkyl or cycloaliphatic hydrocarbon radical which can comprise unsaturations and/or heteroatoms, B″ is a reactive functional group of the same nature as B or B′, n≧1;
R²-A″ (IV)
in which:
R² is a substituted or unsubstituted silicone, linear or branched alkyl, aromatic, arylalkyl, alkylaryl or cycloaliphatic hydrocarbon radical which can comprise one or more unsaturations and/or one or more heteroatoms; and A″ is a reactive functional group of the same nature as A or A′;
the molar ratio I/II being defined as follows:
0.05<I/II
at least one of the entities R or R′ of at least one of the monomers (I) or (II) being aliphatic, cycloaliphatic or arylaliphatic;
R₁ and/or R₂ being polyoxyalkylene radicals.

47. The composition of claim 46, wherein 2≦f≦10.

48. The composition of claim 46, wherein 1≦n≦100.

49. The composition of claim 46, wherein the molar ration of I/II is 0.125≦I/II≦2.

50. The composition of claim 46, wherein the polymerization reactive functional groups A, B, A′ and B′ are carboxyl or amine functional groups.

51. The composition of claim 46, wherein the monomer of formula (I) is a compound in which A represents the amine functional group, B represents the carboxyl functional group, R represents an aromatic radical, and f=2.

52. The composition of claim 31, wherein the proportion of additive A in the powder is between 0.01 and 40% by weight, with respect to the weight of the powder.

53. The composition of claim 31, wherein the thermoplastic powder is a polyamide or a polyester.

54. The composition of claim 53, wherein the thermoplastic powder is polyamide 6, polyamide 6,6, polyamide 11, polyamide 12, polyamides 4,6, 6,10, 6,12, 12,12 and 6,36, their copolymers or their alloys.

55. The composition of claim 31, wherein the powder particles are obtained according to the process comprising the following stages:
a. forming a melt blend of the thermoplastic with at least the polymeric additive in order to obtain a dispersion of discrete particles of the thermoplastic,
b. cooling said blend to a temperature below the softening temperature of the thermoplastic,
c. treating said cooled blend in order to bring about separation of the discrete particles of the thermoplastic.

56. The composition of claim 31, wherein the mean diameter of the powder particles is between 0.1 μm and 1 mm.

57. The composition of claim 31, wherein the mean diameter of the powder particles is between 0.1 and 5 μm.

58. The composition of claim 31, wherein the mean diameter of the powder particles is between 0.5 and 2.5 μm.

59. The composition of claim 31, wherein the proportion of the powder in the composition is between 0.01 and 10% by weight, with respect to the weight of the composition.

60. The composition of claim 31, wherein the composition is in the form of a liquid, solid, or paste.

61. The composition of claim 31, wherein the composition is in the form of a stick, solid or fluid gel, cream, milk, oil, foam, compact or loose powder, emulsion, such as an oil-in-water or water-in-oil emulsion, or an aqueous solution.
